(19) United States
(12) Patent Application Publication
(10) Pub. No.: US 2023/0335269 A1
(43) Pub. Date: Oct. 19, 2023

(54) SPLITTING AND ORDERING BASED LOG FILE TRANSFER FOR MEDICAL SYSTEMS

(71) Applicant: KONINKLIJKE PHILIPS N.V., Eindhoven (NL)

(72) Inventors: Tiblets Zeray DEMEWEZ, EINDHOVEN (NL); Mauro BARBIERI, EINDHOVEN (NL); Serverius Petrus Paulus PRONK, VUGHT (NL); Johannes Henricus Maria KORST, EINDHOVEN (NL)

(21) Appl. No.: 18/027,952

(22) PCT Filed: Sep. 18, 2021
(86) PCT No.: PCT/EP2021/075737

§ 371 Date: Mar. 23, 2023

Related U.S. Application Data
(60) Provisional application No. 63/085,175, filed on Sep. 30, 2020.

(51) Int. Cl. G16H 40/40

(52) U.S. Cl. CPC G16H 40/40

(57) ABSTRACT

A method (100) for transferring at least a portion of a log file to a service center server (16) includes: analyzing the log file to generate notifications (38) of maintenance issues and sending the notifications to the service center server and, for each notification, defining a part of the log associated to the notification; partitioning the log file into parts (40, 42), including the parts associated to the notifications and at least one part corresponding to the remainder of the log file; assigning costs (44) and values (46) to the parts; determining an order of the parts based on the assigned costs and the assigned values for the parts; and transferring the parts to the service center server in the determined order.

### FIELD

The following relates generally to the servicing and maintenance arts, especially as directed to medical imaging device servicing or the servicing of other complex systems, log file analysis arts, and related arts.

### BACKGROUND

A medical imaging system produces log files containing information about the usage and the status of the parts of the system. These files are transferred daily (or on some other time basis) to a data management system where it is processed and stored in a format where it can be consumed for analytics purposes. The files can be large, e.g. on the order of many gigabytes or more for a single day's worth of data. A given hospital or radiology laboratory may have a suite of medical imaging devices each uploading their respective log files, and if the data management system is run by an imaging device vendor then the fleet across all customers of imaging devices can number in the dozens, hundreds, or more imaging devices. This can create substantial bandwidth issues, and consequently, it can take a relatively long time to transfer and process. Apart from the usage and the status of parts, the log files contain error messages, warnings, and additional information from the system. The log files are sent to the data management system with a different timing frequency that mainly depends on the configuration of file transfer in the medical imaging system. Some systems transfer their log files in a daily fashion, while others transfer only at system reboot. The content and size of the log files depend on the usage of the system and the frequency of the file transfer. The transmission time varies depending on the size of the file.

A health monitor is a software application that runs on a medical imaging system (e.g., on the medical imaging device itself, or on a local hospital server computer connected to support the medical imaging device, or so forth) and analyzes log file data to detect certain problems. Typically, the health monitor includes a suite of diagnostic models constructed by experienced engineers to detect specific problems. These diagnostic models may employ analytic expressions, various machine learning (ML) tools trained on historical data, or so forth. The health monitor generates a notification n when a maintenance issue is identified. The health of the system is assessed by monitoring the status of multiple parts and/or subsystems of the medical imaging device to identify potential issues before they actually effect the performance of the system. A generated notification can indicate a possible failure in a certain time window and the severity level. For example, a notification n that is sent at time t(n) may report that a part p(n) of the given system can fail in time interval i(n)=[t(n)+s(n), t(n)+e(n)] with a given probability P(n); In addition, the notification may indicate the severity σ(n) of the failure of p(n) when it occurs. This severity may be indicated by one of: {minor reduced functionality, major reduced functionality, system down}, or any alternative indication approach. Notifications are generated by the health monitor running on or locally with the medical imaging device, and are sent to the data management system for review by a Remote Service Engineer (RSE; also sometimes referred to by other nomenclatures, e.g. a Remote Monitoring Engineer, RME, or so forth). As the amount of data making up a notification is small (e.g. typically on the order of bytes, kilobytes, or at most megabytes) compared with the size of the log files (typically on the order of gigabytes or terabytes), the notifications are assumed to be sent immediately, while log files are sent more slowly, e.g. at their specific frequency (daily, at system reboot, or so forth).

Whenever a medical imaging system runs into issues related to some part of the system, a customer reports the issue to a call center, demanding a rapid solution to prevent the system's reduced functionality or downtime. To further analyze the situation, a remote service engineer (RSE) can analyze recent log files from the system that may contain crucial additional information. Even if no customer report is received, the RSE may analyze the notifications to proactively identify problems in order to provide routine maintenance recommendations to the customer (e.g. hospital or radiology laboratory) or to dispatch a field service engineer to handle the proactive maintenance if appropriate. However, log file availability suffers from various bottlenecks in an end-to-end data pipeline. As a result, the log data pertaining to a particular notification may not be available to the RSE at the time the RSE is reviewing a notification. This can create delays as the RSE must then manually request the relevant log data be uploaded to the data management system; or, the log files are not available for the RSE to analyze the issue and propose a solution.

The following discloses certain improvements to overcome these problems and others.

### SUMMARY

In one aspect, a non-transitory computer readable medium stores a database comprising a log file of at least one medical imaging device. Instructions are readable and executable by at least one electronic processor to perform a method for transferring at least a portion of the log file to a service center server. The method includes: analyzing the log file to generate notifications of maintenance issues and sending the notifications to the service center server and, for each notification, defining a part of the log associated to the notification; partitioning the log file into parts including the parts associated to the notifications and at least one part corresponding to the remainder of the log file; assigning costs and values to the parts; determining an order of the parts based on the assigned costs and the assigned values for the parts; and transferring the parts to the service center server in the determined order.

In another aspect, an apparatus includes a medical imaging device and a database comprising a log file of the medical imaging device. Instructions are readable and executable by at least one electronic processor to perform a method for transferring at least a portion of the log file to a service center server. The method includes: analyzing the log file to generate notifications of maintenance issues and sending the notifications to the service center server and, for each notification, defining a part of the log associated to the notification; partitioning the log file into parts including the parts associated to the notifications and at least one part corresponding to the remainder of the log file; assigning costs and values to the parts; calculating a score for the parts based on the assigned cost and the assigned values; determining an order of the parts based on the calculated scores; and transferring the parts to the service center server in the determined order.

In another aspect, a method for transferring at least a portion of the log file to a service center server includes: analyzing a log file stored in a database of a medical imaging device to generate notifications of maintenance issues and sending the notifications to the service center server and, for each notification, defining a part of the log associated to the notification; partitioning the log file into parts including the parts associated to the notifications and at least one part corresponding to the remainder of the log file; assigning costs and values to the parts; determining an order of the parts based on the assigned costs and the assigned values for the parts; removing overlapping data from later-ordered parts that overlaps with earlier-ordered parts; and transferring the parts to the service center server in the determined order.

One advantage resides in providing expedited transfer of log data pertaining to notifications for a medical system.

Another advantage resides in removing bottlenecks during transfers of log data to a remote server by allocating additional bandwidth for data pertaining to notifications.

Another advantage resides in partitioning log data of a medical system based on generated notifications for the medical system.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for transferring log file data to a service center server in accordance with the present disclosure.

FIG. 2 shows exemplary flow chart operations of the system of FIG. 1.

### DETAILED DESCRIPTION

Typical medical imaging systems can maintain a log of events locally at the imaging machine. Additionally, a software component referred to as a “health monitor” analyzes the log data to detect possible problems. When a possible maintenance issue is detected, a notification is issued and sent to a remote server for review by a RSE.

The health monitor employs various diagnostic models. Each diagnostic model is designed to detect a certain maintenance issue, and the notification contains information including at least identification of the maintenance issue and identification of a subset of the log entries relevant to analysis of the maintenance issue. Additional information in the notification may include: an estimated time window in which the maintenance issue is likely to lead to failure or significant performance degradation; a severity level; and a probability value indicating likelihood of the maintenance issue leading to a failure or significant performance degradation.

However, these notifications often come in groups. For example, notifications may be generated for multiple maintenance issues relating to an aging x-ray tube such as: low tube current; low tube voltage; a time-based notification (e.g. issued at a preset time after the current x-ray tube was installed); and/or so forth.

In current systems, log files can be uploaded to the remote server at defined times, such as at a preset time every day, or only at system reboot. Furthermore, scheduled log data transfer may be adversely impacted by bandwidth bottlenecks. Consequently, when the RSE receives a notification, he or she likely does not have the relevant log data available at the remote server for analysis.

The following discloses an approach for expedited transfer of log data pertaining to notifications. First, the log file is partitioned into parts, designated as parts l₁, l₂, . . . , lₘ. To this end, a given notification n is associated to a subset Sₙ of the total set of log data L making up the log file. This association is done automatically by the diagnostic model that generated the notification, or is based on the identity of the diagnostic model (e.g., for a notification generated by a diagnostic model pertaining to the x-ray tube, the subset Sₙ may include the log file messages relating to the x-ray tube). This subset Sₙ is one of the parts l₁, l₂, . . . , lₘ.

If there are N notifications designated as notifications 1, . . . , N, then these define N parts: S₁, S₂, . . . , S_{N}. An additional part is R(L, N)=L\{S₁∪S₂∪ . . . , S_{N}}, which represents all log entries that are not in any of the subsets S₁, S₂, . . . , S_{N}. Optionally, the (often large) part R(L, N) may be broken into further parts. In one approach, the log file is a list of timestamped log messages, and the part R(L, N) is (further) partitioned by message type (error, warning, information, parameter value, etc.).

Next, the parts l₁, l₂, . . . , lₘ are ordered. In an illustrative approach, each part is assigned a value and a cost. For parts which are subsets S₁, S₂, . . . , S_{N} corresponding to notifications, the value is based on the content of the corresponding notification. For example, value can be based on the notification severity (higher severity biasing to higher value), probability (higher probability biasing to higher value), and time window (more proximate time window biasing to higher value). Parts defined by the partitioning of R(L, N) are usually assigned lower value than any of the parts which are subsets S₁, S₂, . . . , S_{N} corresponding to notifications, and may have values based on the message type (e.g. the parts containing error or warning messages may be assigned higher value than the parts containing information or parameter value messages). The cost of each part is assigned based on its size, e.g. as measured in bytes.

A scoring function ƒ(lᵢ) is then applied to score each part. In one approach, this is simply a ratio of the value v(lᵢ) to the cost c(lᵢ). The parts are then ordered in accord with their respective scores.

After the ordering, any overlapping data is removed. For example, if two parts lᵢ and lⱼ (at least) partially overlap, and if lᵢ is ordered higher than lⱼ, then the overlapping portion is removed from the lower-ordered part (here, lⱼ is replaced by lⱼ−{lᵢ∩lⱼ}). This avoids sending the overlapping portion twice. (Note that if the part lⱼ is identical with or entirely contained in lᵢ then this can result in the part lⱼ being completely eliminated).

Finally, the log data parts are sent to the remote server in the designated order. Preferably, parts of high relevance (e.g., of sufficiently high score) may be sent in an expedited manner by requesting additional bandwidth to get the relevant parts sent quickly. If the log data is to be sent at a predefined time (e.g., 6:00 am), this expediting may also include sending the high relevance data immediately instead of waiting for the predefined time.

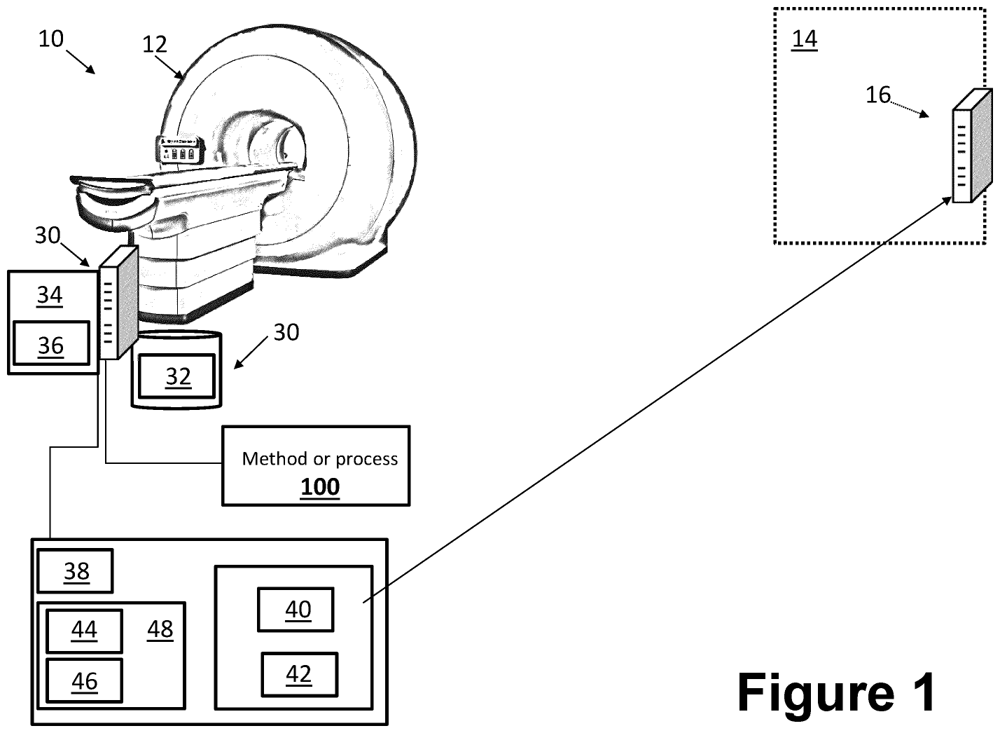

With reference to FIG. 1, an illustrative servicing support system 10 for supporting servicing a device 12 (e.g., a medical imaging device, not shown—also referred to as a medical device, an imaging device, imaging scanner, and variants thereof) is diagrammatically shown. By way of some non-limiting illustrative examples, the medical imaging device 10 may be an interventional X-ray (IXR) system, a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. As shown in FIG. 1, the servicing support system 10 includes, or is accessible by, a service center server 16 in a database backend 14 disposed remote from the device 12.

The device 12 includes a non-transitory computer readable medium comprising a database 30 storing log files 32 related to operation of the device. The log files 32 are generated by the medical imaging device 12, for example by sensors of the medical imaging device, by a controller of the imaging device, and/or so forth. In one commonly used format, the sensors, controller, and so forth generate timestamped messages that form the log files 32. The device 12 also includes a computer or other programmable electronic device 34 running software, such as control software for the illustrative medical imaging device, and also software implementing a health monitor 36 that analyzes the log data to detect possible problems or maintenance issues with the device. When a possible problem is detected, the health monitor 36 generates a corresponding notification 38 based on the data in the log file 32. The electronic device 34 is configured to analyze the notifications 38, and submit an order to the service center server 16 for one or more corresponding parts 40 for the device 12 to address the problem or maintenance issue.

The database 30 store instructions executable by the electronic device 34 to perform a method or process 100 implemented by the servicing support system 10 for transferring at least a portion of the log file 32 via the Internet from the database 30 to the service center server 16. In some examples, the method 100 may be performed at least in part by cloud processing. The method 100 result in an output of an order for one or more parts 40 for the device 12.

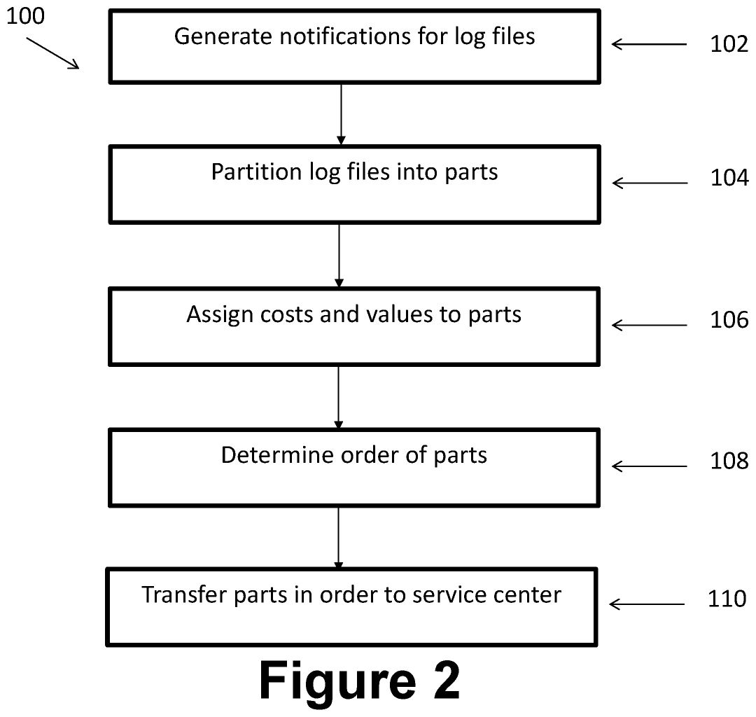

With reference to FIG. 2, and with continuing reference to FIG. 1, an illustrative embodiment of an instance of the method 100 is diagrammatically shown as a flowchart.

At an operation 102, the health monitor 36 of the electronic device 34 is programmed to analyze the log files 32 in the database 30 to generate the notifications 38. More particularly, the health monitor 36 applies various diagnostic models designed to detect various problems. A “problem” in this context can for example be a routine maintenance operation such as replacing a disposable filter at predetermined time intervals, or a part or subsystem that is likely to fail in a near-future time frame, or a part or subsystem that is detected as currently operating sub-optimally but (likely) not yet impacting performance, or a part or subsystem that is detected as currently operating sub-optimally and (likely) degrading performance, or so forth. By way of some nonlimiting illustrative examples, a diagnostic model can be as straightforward as a time-based notification, such as a notification that a filter is scheduled for replacement in two weeks. As another example, a diagnostic model may employ an analytical expression developed by a service engineer, such as detecting when an x-ray tube voltage falls below some specified threshold which the expert engineer has recognized is suggestive that the x-ray tube is reaching end-of-life. As another example, a diagnostic model may employ an artificial neural network (ANN), support vector machine (SVM), regression classifier, or other machine learning (ML) component trained on historical data to detect a likely occurrence of a problem, and optionally outputting a probability of the problem occurring in a specified time frame. For each notification 38, a part of the log file 32 is defined as being associated to the corresponding notification. For example, if the diagnostic model issuing the notification pertains to a particular part or subsystem of the medical imaging device 12, then the part of the log file defined as being associated to the notification may be the messages of the log file generated by that part or subsystem and/or sensor measurements associated with that part/subsystem. The notifications 38 are generated by maintenance issue-specific algorithms implemented on the health monitor 36.

At an operation 104, the log files 32 are partitioned into the parts 40. In one example, the log files 32 can be partitioned into the parts 40 associated to the corresponding notifications 38 (i.e., “assigned parts” 40). In some examples, at least one part 42 of the log file 32 corresponding to a remainder of the log file (i.e., not associated to any of the notifications 38) is partitioned from the log file 32. To do so, the remainder of the log file 32, which comprises a list of timestamped log messages, is partitioned based on message type (e.g., error, warning, information, parameter value, etc.) to determine the unassigned part 42.

At an operation 106, costs 44 and values 46 are assigned to the parts 40 and 42. In some embodiments, the costs 44 of the parts 40 can be determined based on the sizes (e.g., in bytes) of the parts 40 of the log file 32. In some embodiments, the values 46 of the parts are determined based on content of the corresponding notifications 38. In some examples, the content of the notifications 38 comprises severities of the notifications. In this example, the assigning operation 106 includes determining the values 46 assigned to the assigned parts 40 associated to the notifications based at least in part on the severities of the corresponding notifications. The severity levels can be arranged based on any suitable ranking (e.g., a higher severity having a higher value, a higher severity being indicative of a maintenance issue that can happen in a short time frame, and so forth). In another example, the content of the corresponding notification 38 comprises a probability of the notification, and the values 46 of the assigned parts 40 are determined based at least in part on the probabilities. In a further example, the content of the corresponding notification 38 comprises a time window of the notification, and the values 46 of the assigned parts 40 are determined based on the time window, in which the assigned parts 40 having time window that is shorter than other time windows have a higher value. The unassigned parts 42 are assigned a value 46 that is lower than a lowest value assigned to any of the assigned parts 40 associated to a notification 38.

At an operation 108, an order of the parts 40 and 42 is determined based on the assigned costs and assigned values for the parts. To do so, a score 48 is calculated for the assigned parts 40 and unassigned parts 42, and the parts are ordered based on the calculated scores. The score 48 can be calculated by calculating a ratio of the values 46 and the costs 44.

At an operation 110, the parts 40 and 42 are transferred to the service center server 14 in the determined order. In some examples, parts 40, 42 having a score 48 that exceeds a predetermined score threshold are transferred using an expedited transfer protocol (i.e., the higher scored parts are transferred “immediately” so that the corresponding components for the device 12 can be ordered more quickly). In one approach, this can be done by requesting additional bandwidth to get the relevant parts sent quickly. In other examples, after the determining of the order and before the transferring, overlapping data from later-ordered parts 40, 42 that overlaps with earlier-ordered parts are removed, so that identical components are not repeatedly ordered.

### Example

The following provides a more detailed example of the method 100. The notifications 38 are generated by maintenance issue-specific algorithms implemented in the health monitor 36. In this example, the device 12 comprises of an interventional X-ray (IXR) system with an X-ray tube near failure. The algorithm of the health monitor for this might be as follows:

Tube failure probability=function(time_since_installation, average operating voltage, average operating tube current)

where average operating voltage might be averaged all operations of the X-ray tube since its installation, and likewise for average operating tube current.

If tube failure probability is >T1 then a “Replace X-ray tube soon” notification is issued. Some parameters of the “Replace X-ray tube soon” notification might be: Tube failure probability (the actual value); a Severity (a high value since tube failure shuts down the IXR until it is replaced) and Projected failure time interval, which can be given by:

failure time interval i_{T1(n)}=[t(n)+sᵣ₁₍ₙ₎,t(n)+en_{T1(n)}]

where t(n) is the current time and the interval s(n)-e(n) is the expected time interval of failure referenced to current time t(n). s(n) and e(n) may be constants based on historical data that are hard-coded into the algorithm or stored in a configuration file of the health monitor 36.

A subset Sₙ of the log file L that is associated to the “Replace X-ray tube soon” notification would be all messages in the log that are relevant for assessing the X-ray tube health, such as tube voltage and current measurement messages, the message indicating when the X-ray tube was installed and any associated messages such as indicating the make/model of the installed X-ray tube etc.

Once the notifications 38 are generated by the health monitor 36, the log file 32 can be partitioned and ordered correspondingly. Hence, both the partitioning of the log file 32 into the parts 40 and the ordering of these parts may depend on the health monitor 36.

The log file L can be arranged as an ordered set of log messages. For a given log file L and for each possible notification n, a corresponding subset of the log messages S(n)⊆L in the given log file is indicated as being relevant for notification n. All log messages in S(n) are assumed to form one part in the subsequent ordering of parts. For two notifications n and n′ it may be the case that S(n) and S(n′) have some overlap. If only one notification n is generated by the health monitor, then log file L is split into two parts S(n) and L\S(n). Likewise, if two notifications n and n′ are generated, then then L is partitioned into S(n), S(n′) and L\{S(n) U S(n′)}. To avoid that the overlap between S(n) and S(n′) can be sent twice, any duplicates after ordering the parts can be removed. For example, if S(n) is sent first, then the overlap between S(n) and S(n′) can be removed from S(n′), before it is transmitted. N(t) denotes the set of all notifications that have been generated by the health monitor at time t. For convenience, R(L, N) denotes the log messages in L that do not occur in any of the sets S(n), n∈N.

In addition to the partitioning, a value and cost is assigned to the parts. A cost is assigned to the parts based on bandwidth or computing power and a value is assigned based on the content of the health monitor notification. As an alternative, different priorities can also be assigned to the log messages within one subset S(n), resulting in a more fine-grained partitioning of the log file. In practice, a diagnostic model that uses S(n) as input can probably only be able to carry out a meaningful analysis if all the log messages in S(n) are available.

Once the log files are partitioned, the parts of the log file can be ordered. If a high-severity notification n is generated at time t with a corresponding time interval i(n)=[t(n)+s (n), t(n)+e(n)], for which the time e(n) to the deadline t(n)+e(n) is relatively small, then the related log file part(s) can be transmitted first and additional bandwidth can be requested to get the relevant data transmitted as soon as possible.

If two or more notifications are generated by the health monitor 36, all with the same severity and time interval, then the related log file parts 40 can be ordered by decreasing probability, such that the information that is relevant for the most probable issues are received first.

If two or more notifications are generated by the health monitor 36, all having the same severity, time interval and probability, then the related log file parts can be ordered by decreasing size, such that the time that one of the predictive models can start executing is as small as possible.

The above alternatives merely serve as examples of possible orderings that are adapted to the number and characteristics of the notifications generated by the health monitor. Other, more advanced orderings can readily be conceived and more complex cases of multiple, more diverse notifications with distinct severity, probability or time interval properties can be handled correspondingly.

In general, the ordering of the parts l₁, . . . , lₘ uses an ordering function ƒ that assigns a score ƒ(lᵢ) to each part lᵢ, such that the parts can be sent in order of decreasing score. A sensible way to order the parts is dividing the value v(lᵢ) by the cost c(lᵢ), i.e., to have

$$f ( {l}_{i} ) = \frac{v ( {l}_{i} )}{c ( {l}_{i} )}$$

Clearly, alternative orderings are possible, by adapting the definition of the value v(lᵢ) or by changing the ordering function appropriately.

Subsequently, a cost is assigned to the parts based on bandwidth or computing power and a value is assigned based on the content of the health monitor notification. Having the cost and value, parts of the file are ordered and transferred to the data management system. In this way, more relevant data is transferred earlier than less relevant data. The less relevant data can be transmitted, following a common file transfer procedure.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
a database comprising a log file of at least one medical imaging device;
instructions readable and executable by at least one electronic processor to perform a method for transferring at least a portion of the log file to a service center server, the method comprising:
analyzing the log file to generate notifications of maintenance issues and sending the notifications to the service center server and, for each notification, defining a part of the log associated to the notification;
partitioning the log file into parts, including the parts associated to the notifications and at least one part corresponding to the remainder of the log file;
assigning costs and values to the parts;
determining an order of the parts based on the assigned costs and the assigned values for the parts; and
transferring the parts to the service center server in the determined order.

2. The non-transitory computer readable medium of claim 1, wherein the assigning includes:
determining the costs of the parts based on sizes of the parts.

3. The non-transitory computer readable medium of claim 1, wherein the assigning includes:
determining the values of the parts associated to the notifications based on content of the corresponding notifications.

4. The non-transitory computer readable medium of claim 3, wherein the content of the corresponding notifications comprises severities of the notifications, and wherein the assigning includes:
determining the values assigned to the parts associated to the notifications based at least in part on the severities of the corresponding notifications.

5. The non-transitory computer readable medium of claim 3, wherein the content of the corresponding notification comprises a probability of the notification, and wherein the determining of the values includes:
determining the values assigned to the parts associated to the notifications based at least in part on the probabilities of the notifications.

6. The non-transitory computer readable medium of claim 3, wherein the content of the corresponding notification comprises a time window of the notification, and wherein the determining of the values includes:
determining the value of the parts based on the time window, wherein parts having time window that is shorter than other time windows have a higher value.

7. The non-transitory computer readable medium of claim 3, wherein the at least one part corresponding to the remainder of the log file is assigned a value that is lower than a lowest value assigned to any part associated to a notification.

8. The non-transitory computer readable medium of claim 1, wherein the partitioning includes partitioning the remainder of the log file into multiple parts based on message type.

9. The non-transitory computer readable medium of claim 1, wherein the method further comprises:
after the determining of the order and before the transferring, removing overlapping data from later-ordered parts that overlaps with earlier-ordered parts.

10. The non-transitory computer readable medium of claim 1, wherein the determining of the order includes:
calculating scores for the parts; and
ordering the parts based on the calculated scores.

11. The non-transitory computer readable medium of claim 2, wherein the calculating includes:
calculating a ratio of the values and the costs for the parts.

12. The non-transitory computer readable medium of claim 10, wherein the transferring includes:
transferring parts having a score exceeding a predetermined score threshold using an expedited transfer protocol.

13. An apparatus, comprising:
a medical imaging device;
a database comprising a log file of the medical imaging device;
instructions readable and executable by at least one electronic processor to perform a method for transferring at least a portion of the log file to a service center server, the method comprising:
analyzing the log file to generate notifications of maintenance issues and sending the notifications to the service center server and, for each notification, defining a part of the log associated to the notification;
partitioning the log file into parts, including the parts associated to the notifications and at least one part corresponding to the remainder of the log file;
assigning costs and values to the parts;
calculating a score for the parts based on the assigned cost and the assigned values;
determining an order of the parts based on the calculated scores; and
transferring the parts to the service center server in the determined order.

14. The apparatus of claim 13, wherein the assigning includes:
determining the costs of the parts based on sizes of the parts.

15. The apparatus of claim 13, wherein the assigning includes:
determining the values of the parts associated to the notifications based on content of the corresponding notifications;
wherein the at least one part corresponding to the remainder of the log file is assigned a value that is lower than a lowest value assigned to any part associated to a notification.

16. The apparatus of claim 13, wherein the partitioning includes partitioning the remainder of the log file into multiple parts based on message type.

17. The apparatus of claim 13, wherein the method further comprises:
after the determining of the order and before the transferring, removing overlapping data from later-ordered parts that overlaps with earlier-ordered parts.

18. The apparatus of claim 13, wherein the calculating includes:
calculating a ratio of the values and the costs for the parts.

19. The apparatus, of claim 17, wherein the transferring includes:
transferring parts having a score exceeding a predetermined score threshold using an expedited transfer protocol.

20. A method for transferring at least a portion of the log file to a service center server, the method comprising:
analyzing a log file stored in a database of a medical imaging device to generate notifications of maintenance issues and sending the notifications to the service center server and, for each notification, defining a part of the log associated to the notification;
partitioning the log file into parts, including the parts associated to the notifications and at least one part corresponding to the remainder of the log file;
assigning costs and values to the parts;
determining an order of the parts based on the assigned costs and the assigned values for the parts;
removing overlapping data from later-ordered parts that overlaps with earlier-ordered parts; and
transferring the parts to the service center server in the determined order.
